(19) United States
(12) Patent Application Publication
(10) Pub. No.: US 2017/0112441 A1
(43) Pub. Date: Apr. 27, 2017

(54) GARMENT FOR MAINTAINING WARMTH WHILE FACILITATING THE PLACEMENT OF A MONITORING DEVICE

(71) Applicant: Susan DONOHOE, Elverson, PA (US)

(72) Inventor: Susan DONOHOE, Elverson, PA (US)

(21) Appl. No.: 15/333,807

(22) Filed: Oct. 25, 2016

Related U.S. Application Data
(60) Provisional application No. 62/246,910, filed on Oct. 27, 2015.

(51) Int. Cl. A61B 5/00; A61B 5/1455

(52) U.S. Cl. CPC A61B 5/6807; A61B 5/14552; A61B 5/6829; A61B 5/6826

(57) ABSTRACT

A garment includes body portions for overlying at least portions of an anatomical feature, to keep the anatomical feature warm, an opening formed in the body portions, and flexible portions associated with the opening in the body portions. The flexible portions allow portions of the garment to be folded away from an initial position on the anatomical feature, and to be returned to the initial position to cover the selected portions of the anatomical feature. The flexible portions of the garment facilitate placement and positioning of a probe, sensor or other monitoring equipment to be placed on selected portions of the anatomical feature while maintaining the warmth of the anatomical feature.

### CROSS-REFERENCE TO RELATED APPLICATIONS

This application claims priority from U.S. Provisional Patent Application No. 62/246,910, titled “Garment for Maintaining Warmth While Facilitating the Placement of a Monitoring Device”, filed on Oct. 27, 2016, the entire contents of which is incorporated by reference herein.

### BACKGROUND OF THE INVENTION

This invention primarily relates to a garment, such as a sock, glove or similar flexible covering, that can be used to maintain the warmth of an appendage, primarily an extremity, while facilitating the placement of a monitoring device, such as medical monitoring equipment, on the appendage or extremity.

Any of a number of procedures require monitoring equipment to be applied to an appropriate portion of the body for purposes of determining desired parameters and conditions. In the discussion which follows, primary emphasis will be placed on the application of a pulse oximeter to the foot of an infant or small child. However, it is to be understood that such improvements can similarly be used for the application of other medical or general purpose equipment to other portions of the body, for users other than infants and small children, such as preemies (i.e., a baby born prematurely), youth and adults.

Pulse oximetry provides a simple, non-invasive, reasonably accurate estimation of arterial oxygen saturation. Such techniques can be used to both quickly and painlessly monitor such conditions, for example, to screen for certain heart defects in a baby, as well as respiratory care, emergency room use, in pediatric wards and intensive care units, and for perioperative care. In use, a suitable probe associated with a pulse oximeter is preferably applied to a toe of an infant or a small child, similar to the manner in which an oxygen sensor is clamped to the end of a finger for a youth or an adult. The applied probe is then used to transmit signals to the corresponding pulse oximeter to suitably monitor conditions in otherwise known and conventional fashion.

In practice, while pulse oximetry provides a useful tool for monitoring various conditions, the use of such equipment on infants and small children has been found to present certain challenges that can at times limit the utility of such devices. For example, to accommodate placement of the probe and passage of the wires connecting the probe and the corresponding monitoring device, it is common practice to leave the foot exposed to avoid interference with the monitoring equipment. This, however, limits the ability to keep the foot warm when the probe is in use, even in the presence of ambient heating equipment. This is particularly important for a newborn or a preemie because the potential for heat loss is significantly greater in such patients. Another consideration is the potential for ambient light to interfere with readings taken with the oximeter probe.

Various coverings have been developed for overlying at least portions of a foot, and in some cases a hand, to facilitate the placement of a pulse oximeter probe, or for stabilizing the position of a placed pulse oximeter probe. Known coverings have not sufficed for satisfactorily covering the foot of an infant or a small child without compromising operations of the associated monitoring equipment.

Many of these coverings are configured to expose the toes, or the fingers if applied to a hand, through various types of openings. For example, U.S. Patent Application Publication No. 2007/0244377 (Cozad et al.) discloses a sleeve that can be applied over the foot or hand of a patient, exposing the toes or fingers to ensure visual confirmation of blood perfusion through tissue. Other open-toed embodiments are disclosed in U.S. Pat. No. 5,623,734 (Pugliatti), U.S. Pat. No. 1,999,929 (Hearn) and U.S. Design Pat. No. Des. 112,368 (Hynes). This would also include an embodiment disclosed in U.S. Design Pat. No. D707,036 (Patterson), which has an exposed toe configuration having an upwardly directed opening exposing the instep of the foot while being fully enclosed along the arch.

Other coverings are configured to enclose the toes, or the fingers if applied to a hand. This would include fully enclosed embodiments, for example, the embodiments disclosed in U.S. Pat. No. 7,433,726 (Perkins), U.S. Pat. No. 6,470,199 (Kopotic et al.) and U.S. Pat. No. 5,842,982 (Mannheimer). This would also include partially enclosed embodiments, for example, the embodiment disclosed in U.S. Pat. No. 6,047,201 (Jackson, III). This would further include an embodiment disclosed in U.S. Pat. No. 3,329,972 (Schwab et al.), which can be switched between an open and a fully enclosed position using a foldable toe piece.

None of the known coverings were satisfactory for effectively covering the foot of an infant or a small child without compromising operations of the associated monitoring equipment, leaving a need for the development of a covering capable of satisfying these requirements.

### BRIEF SUMMARY OF THE INVENTION

In accordance with the present invention, such requirements are achieved by providing a garment which can, to the extent possible, overly an anatomical feature such as an appendage, and in particular an extremity, to keep the anatomical feature warm. An opening is provided to allow portions of the garment to be folded down or pulled away, to allow a probe or a sensor associated with desired monitoring equipment to be applied to selected portions of the anatomical feature or to be adjusted, as needed, and to be returned to the initial position to cover the selected portions of the anatomical feature while allowing the probe or sensor to freely communicate with associated monitoring equipment.

The garment can in this way be effectively used to allow a probe or a sensor associated with desired monitoring equipment to be placed on an appendage, primarily an extremity such as a foot or a hand, while covering the anatomical feature to the extent possible to keep the feature warm while the probe or sensor is being worn by the user. This provides a garment that can significantly improve wearer comfort, while allowing full functionality of the associated monitoring equipment. This, in turn, permits quick and efficient placement of the probe or sensor on the wearer while allowing selected portions of the garment to be pulled back for desired reasons, such as placement or adjustment of the probe or sensor. Moreover, the garment can be left on the wearer, even when the associated monitoring equipment is not in use, maintaining warmth and improving time on task for a professional or caretaker by avoiding the need to remove and replace the garment. Covering the probe or sensor can also help avoid ambient light interference, and can facilitate checking for skin pigmentation and nail color.

This allows a pulse oximeter probe or sensor to be conveniently placed on an appendage, primarily on a foot or a hand, at a desired location which is appropriate for a particular use or application. Placement of the pulse oximeter probe or sensor on the wearer's foot can also make it significantly more difficult for the infant or small child to grasp, pull on or remove the probe or sensor following placement. The garment is further configured to permit work to be done around the ankle or wrist of the wearer, in addition to facilitating identification of the wearer, blood work or placement of other instruments at the ankle or wrist. The garment can also be provided with non-skid surfaces or features to allow the user to walk while wearing the garment.

For a further description of garments in accordance with the present invention, reference is made to the detailed description of preferred embodiments of the invention, provided below, together with the following drawings.

### BRIEF DESCRIPTION OF THE DRAWINGS

The following detailed description of preferred embodiments of the invention will be better understood when read in conjunction with the appended drawings. It should be understood, however, that the disclosure is not limited to the precise arrangements and instrumentalities shown. In the drawings:

FIG. 1 is an isometric view of the garment of the present invention, placed on a foot;

FIG. 2 is a side elevational view of the garment shown in FIG. 1;

FIG. 3 is a bottom plan view of the garment shown in FIG. 1; and

FIG. 4 is a plan view of the bottom of an alternative embodiment of the garment of the present invention, including non-skid features.

### DETAILED DESCRIPTION OF THE INVENTION

Certain terminology is used in the following description for convenience only and is not limiting. The words “lower,” “bottom,” “upper” and “top” designate directions in the drawings to which reference is made. The words “inwardly,” “outwardly,” “upwardly” and “downwardly” refer to directions toward and away from, respectively, the geometric center of the garment and designated parts thereof, in accordance with the present disclosure. Unless specifically set forth herein, the terms “a,” “an” and “the” are not limited to one element, but instead should be read as meaning “at least one.” The terminology includes the words noted above, derivatives thereof and words of similar import.

It should also be noted that the terms “first,” “second,” “third” and the like may be used herein to modify various elements. These modifiers do not imply a spatial, sequential, or hierarchical order to the modified elements unless specifically stated. It should also be understood that the terms “about,” “approximately,” “generally,” “substantially” and like terms, used herein when referring to a dimension or characteristic of a component of the invention, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

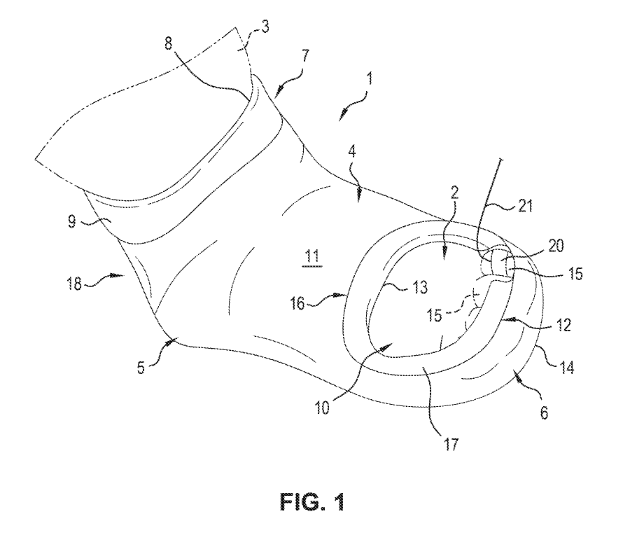

Referring to the drawings in detail, wherein like numerals indicate like elements throughout, there is shown in FIGS. 1-4 a garment in accordance with the present invention which is being used for the application of a pulse oximeter to the toe of an infant or small child. The illustrated garment is implemented as a sock 1 worn on the foot 2 of an infant 3. It is to be understood that the embodiments illustrated in FIGS. 1-4 constitute only some of the various ways that the improvements of the present invention can be used for the application of medical or general purpose equipment to various portions of the body, for users other than infants or small children. For example, the garment of the present invention can similarly be used to apply a pulse oximeter to a hand, to a wrist or to an ankle of the infant 3, or to apply other types of probes, sensors or equipment to one or more appendages or extremities, or to users other than infants, including preemies, newborn babies, toddlers, children, adolescents and adults, as well as some veterinary applications.

In practice, there are different factors that determine the appropriate placement for a pulse oximeter on a patient. Primarily, the site is chosen for the measurement of a blood oxygen saturation level (SpO₂) and must be adequately perfused with blood. This can include locations such as the toe of a foot or the finger of a hand, an outer aspect of the palm of a hand or the sole of a foot, or potentially on an ear or on the Achilles tendon. Other factors that can determine the appropriate placement can include, among others, the presence of nail polish or burns, blood flow, digit size, age of the patient and any medical issues. These decisions are normally made by medical personnel considering the patient and the use or application involved. The purpose of the garment of the present invention is to best accommodate the patient, irrespective of where the placement is to occur, either at the previously mentioned sites, or at new sites that may become useful in the future. Consequently, although the following discussion will proceed in the context of a sock 1 used for the application of a pulse oximeter to the toe of an infant, for convenience of description only, similar coverings can be implemented in different configurations to mate with desired anatomical features of the body, as desired for a particular application.

The sock 1 includes a body 4 for overlying the foot 2 and is preferably configured to conform to the features of the wearer. The sock 1 can be implemented as a tube sock, or as a formed sock having a fitted heel portion 5 and a fitted toe portion 6, as desired. Upper portions 7 of the body 4 include an opening 8 for receiving the foot 2. The upper portions 7 of the body 4 preferably include an elastic band 9 which fully surrounds the opening 8 to help maintain the sock 1 in desired position and to maximize the warmth obtained when wearing the sock 1.

In accordance with the present invention, toe portions 6 of the sock 1 are provided with an opening 10, which preferably extends from the toe portion 6 to the instep 11 of the foot 2. The size and shape of the opening 10 can be varied, as desired, although a round or oval shaped opening is presently considered preferred. Forward, i.e., distal, portions 12 of the perimeter 13 of the opening 10 are preferably spaced from the tip 14 of the sock 1 to optimize coverage of the toes 15 of the foot 2. Rearward, i.e., proximal, portions 16 of the perimeter 13 of the opening 10 are preferably minimized, to the extent possible, to further optimize coverage of the instep 11 of the foot 2. The perimeter 13 of the opening 10 preferably includes an elastic band 17 which fully surrounds the opening 10 to help maintain the sock 1 in the desired position and to maximize the warmth obtained when wearing the sock 1.

The sock 1 is preferably soft, compliant and flexible, to maximize comfort, and can be made from any of a variety of conventionally available materials. A blend of materials is preferred to achieve this, and to maximize warmth, one example being a blend of cotton, nylon and spandex, which can further include rubber, if desired. A blend of approximately 75% cotton, approximately 21% nylon, approximately 3% spandex and approximately 1% rubber is presently considered particularly preferred. However, it is to be understood that the material used can be a blend of the foregoing materials, with varying proportions, or other materials in proportions suitable for a particular use. The selected material or blend of materials is preferably machine washable to permit frequent cleaning. The sock 1 can be any color, or any combination of colors, and can include any textual material, logo's or designs considered desirable for appearance or needed to comply with consumer or other regulations.

In use, the toes 15 of the foot 2 are introduced through the opening 8 of the sock 1 and the body 4 of the sock 1 is drawn over the foot 2, in otherwise conventional fashion. The toes 15 of the foot 2 will then be located within the toe portion 6 of the sock 1 so that the forward portions 12 of the opening 10 substantially cover the toes 15. The body 4 of the sock 1 will then cover substantial portions of the foot 2, preferably from the instep 11 of the foot 2 to a point above the ankle 18. The other foot of the wearer (not shown) can also receive a sock, which preferably corresponds to the sock 1 to allow the toes of either foot to be accessed for purposes of receiving desired equipment. As an alternative, a conventional sock can be used to cover the other foot, if desired.

When desired, for purposes of connecting equipment, for adjusting equipment that has already been connected, or for other similar purposes, forward portions 12 of the perimeter 13 of the opening 10 are grasped and drawn over the toes 15. After clearing the toes 15, the grasped portions of the perimeter 13 can be released, allowing the toe portion 6 to pass beneath the foot 2 and leaving the toes 15 freely accessible. Remaining portions of the foot 2 continue to be covered by the sock 1 helping to maintain warmth as a desired procedure is being performed, in the illustrated example, the placement of a probe 20 of a pulse oximeter on one of the toes 15 of the foot 2, in this illustration, the small toe. Following this, the toe portion 6 can be grasped, drawn over the toes 15, and returned to its initial position covering the toes 15, as desired. Any leads 21 or other connections associated with the probe 20, or other device if used, can then freely pass through the opening 10. Such movements can be facilitated by the elastic band 17 associated with the opening 10, if used, or the elastic nature of the fabric which preferably forms the sock 1, as desired. The foregoing procedures can be repeated, as often as is needed, to achieve desired monitoring, testing or some other desired procedure.

As a result, the foot 2 is covered and kept warm while allowing a caretaker to safely and quickly don and use a pulse oximeter. This promotes patient comfort by keeping the foot 2 warm while allowing for quicker and safer delivery of services by medical staff. Similar benefits are achievable for use in homecare, in hospitals, in emergency departments and intensive care units, for both out-patient and in-patient facilities.

The sock 1 preferably sits low on the ankle 18 to continue and facilitate the existing practice of using ankle bracelets for purposes of identification, and to facilitate blood testing at the ankle. The opening 10 is preferably sufficiently large to allow the toe portion 6 to be peeled back to facilitate the application of probes, sensors, monitors and other desired equipment. The sock 1 can also be pushed up along the ankle 18, without being removed, facilitating the testing of reflexes and desired massage therapy.

The sock 1 is particularly useful for the application of medical equipment to infants and children. It is common for such patients to pull at any cords or other connections within their grasp, and conventional hospital socks were often placed over the hands of a patient to prevent this. The need for such measures is eliminated by the use of a sock 1 placed on the foot of the patient, out of reach of the patient's hands. The sock 1 also eliminates the need to doff and don garments to perform a desired treatment or procedure. For example, pulse oximetry or the placement of an intravenous line in a patient's foot is facilitated by allowing connecting wires or lines to freely pass through the opening 10, allowing the sock 1 to fit over such equipment and conform to the foot 2. This also facilitates the placement of arterial lines in the feet, as well as quick access to the toes 15 for purposes of assessment. For example, after a leg injury, the sock 1 allows the toes 2 to be quickly assessed for compartment syndrome by allowing temperature, coloring and feeling in the toes 2 to be easily checked on a desired periodic basis, and especially after a new injury. Surgeries can also be facilitated; for example, surgeries requiring neurovascular checks in the feet, as well as cath lab, interventional radiology and other procedures requiring a doctor to access the femoral artery. The sock 1 can also be used to facilitate ankle joint injections and the palpation of foot pulses.

The sock 1 can also be worn as a primary sock, even when not being used for a particular treatment or other purpose, and does not require a change into a hospital sock if the patient is getting up and walking. Non-skid features, such as the elastomeric dimples 23 shown in FIG. 4, can be provided to help prevent slips and falls. Wearing the sock 1 also helps keep a probe, sensor or a connecting wire or line in place and can help avoid ambient light interference with the equipment being used, as well as being safe for use in a medical inspection room (MIR).

It will be understood that while the present invention has been described based on specific embodiments incorporating specified parts, the present invention further encompasses all enabled equivalents of the parts described, and that various changes in the details, materials and arrangement of parts which have been herein described and illustrated in order to explain the nature of this invention may be made by those skilled in the art within the principle and scope of the invention as expressed in the claims which follow.

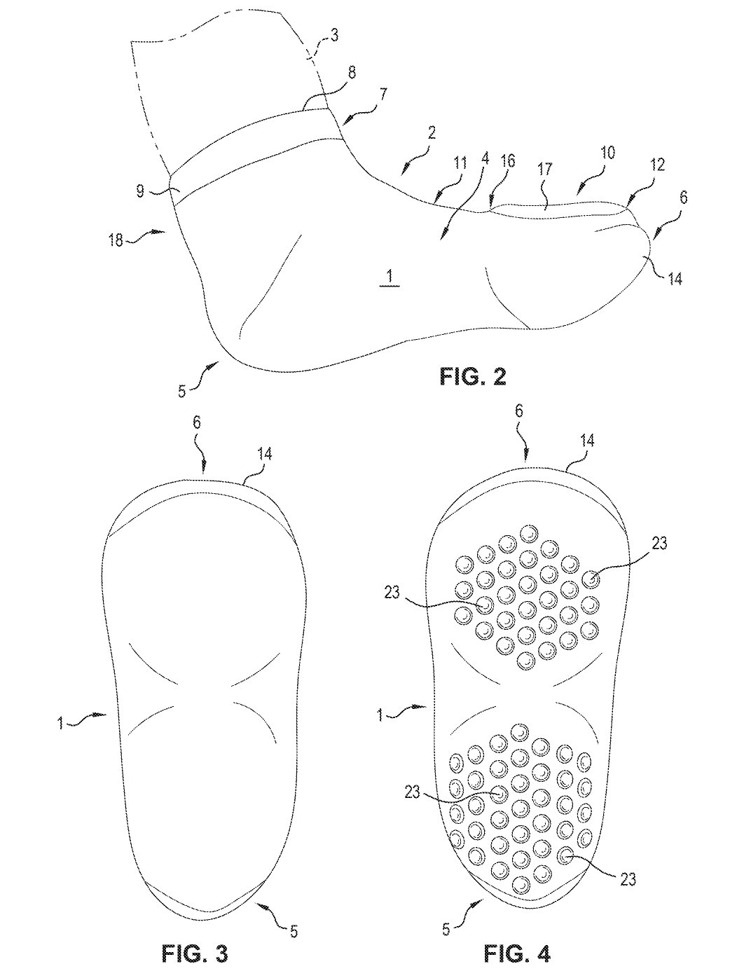

For example, the sock 1 is shown with a completed opening 8 at the ankle 18 and a completed opening 10 over the instep 11 of the foot 2. As an alternative, the openings 8, 10 can be provided with seams or flaps that can be separated to allow the sock 1 to be wrapped around the ankle 18 or the instep 11, and various closures can be used to join the seams or flaps such as snaps, buttons, hooks, zippers or Velcro® closures. Other openings in the sock 1, in addition to the opening 10, can also be provided, if desired. The opening 10 or any additional openings provided in the sock 1 either can be left open, as shown in FIGS. 1 and 2, or can be provided with a flap for selectively enclosing the opening, if desired.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A garment comprising:
a body for substantially overlying and conforming to an anatomical feature of a subject to keep the anatomical feature warm, the body including an opening formed therein, the opening defining an elastic perimeter for selective stretching of the opening,
wherein portions of the body of the garment distal from the opening are selectively movable from (i) a covering position, wherein the portions of the body cover distal members of the anatomical feature to (ii) an exposing position, wherein at least one of the distal members of the anatomical feature projects through the opening and is exposed, such that a probe or sensor associated with desired monitoring equipment is placeable on selected portions of the at least one distal member of the anatomical feature,
the portions of the body of the garment distal from the opening being selectively returnable to the covering position thereof.

2. The garment of claim 1, wherein the probe or sensor includes a connection, and wherein the connection extends through the opening and communicates with associated monitoring equipment.

3. The garment of claim 2, wherein the monitoring equipment is a pulse oximeter.

4. The garment of claim 1, wherein the anatomical feature is an appendage.

5. The garment of claim 4, wherein the anatomical feature is an extremity associated with the appendage.

6. The garment of claim 5, wherein the appendage is a foot and the extremity is a toe.

7. The garment of claim 1, wherein the garment is formed of a soft, compliant and flexible material comprised of a blend of materials including cotton, nylon and spandex.

8. The garment of claim 7, wherein the blend further includes rubber.

9. The garment of claim 8, the blend being comprised of approximately 75% cotton, approximately 21% nylon, approximately 3% spandex and approximately 1% rubber.

10. The garment of claim 1, wherein the garment is a tube sock.

11. The garment of claim 1, wherein the garment is a formed sock.

12. The garment of claim 1, wherein at least portions of the opening include an elastic band at least partially surrounding the opening.
